(19) United States
(12) Patent Application Publication
(10) Pub. No.: US 2014/0125969 A1
(43) Pub. Date: May 8, 2014

(54) APPARATUS AND METHODS FOR PERFORMING OPTICAL TOMOGRAPHY ON DOSIMETERS FOR CALIBRATING RADIOTHERAPY EQUIPMENT

(71) Applicant: Kevin James Jordan, London (CA)

(72) Inventor: Kevin James Jordan, London (CA)

(73) Assignee: LAWSON HEALTH RESEARCH INSTITUTE, London (CA)

(21) Appl. No.: 14/070,761

(22) Filed: Nov. 4, 2013

Related U.S. Application Data
(60) Provisional application No. 61/721,697, filed on Nov. 2, 2012.

(51) Int. Cl. G01T 1/06; A61N 5/00; G01N 21/59

(52) U.S. Cl. CPC G01T 1/06; G01N 21/59; A61N 5/00
USPC 356/72; 356/432

(57) ABSTRACT

An apparatus and method for performing tomography are disclosed herein. The apparatus includes an emitter for scanning an object with a detection beam, a diffuser for scattering a transmitted portion of the detection beam that passes through the object in order to generate a scattered signal, and at least one detector for detecting a portion of the scattered signal. The diffuser has a diffusive surface area, and the detector has a total detection area that is smaller than the diffusive surface area.

### RELATED APPLICATIONS

This application claims the benefit of U.S. Provisional Patent Application Ser. No. 61/721,697 filed Nov. 2, 2012 and entitled “Apparatus and Methods for Performing Optical Tomography on Dosimeters for Calibrating Radiotherapy Equipment”, the entire contents of which are hereby incorporated by reference herein for all purposes.

### TECHNICAL FIELD

One or more embodiments herein relate to apparatus and methods for performing tomography, and in particular, performing optical tomography on dosimeters for calibrating radiotherapy equipment.

### INTRODUCTION

Optical tomography is a form of computed tomography that creates a digital model of an object by reconstructing images made from light transmitted and scattered through the object. This technique is frequently used in healthcare, particularly for imaging soft tissues.

Another use in healthcare is calibrating radiotherapy equipment prior to treating a patient. In such cases, a transparent or translucent object, called a “dosimeter”, may be irradiated with a test dosage of radiation from the radiotherapy equipment. Some dosimeters are made of a material that experiences a change in optical transmittance (e.g., turns opaque) when irradiated. Thus, it is possible to model the effects of the test dosage by scanning the dosimeter using optical tomography to identify which locations of the dosimeter have experienced a change in optical transmittance.

A common difficultly with optical tomography is that light scatters when it impinges the object. This scattering can make it difficult to accurately detect the amount of light transmitted through each part of the object. To counteract the effect of scattering, conventional optical tomography techniques rely upon methods to reduce or minimize detection of scattered light, for example, by using more transparent dosimeters, geometries with smaller acceptance angles (i.e. longer and smaller field of views), apertures or pinholes, telecentric geometries, and the like. Furthermore, optical imaging lenses are often placed behind the object in order to focus the light that has passed through the dosimeter onto small optical sensors. Unfortunately, these optical lenses can be prohibitively expensive, especially as their size increases.

### BRIEF DESCRIPTION OF THE DRAWINGS

The drawings included herewith are for illustrating various examples of articles, methods, and apparatuses of the present specification. In the drawings:

FIG. 1 is a schematic diagram of an apparatus for performing tomography of an object according to one embodiment;

FIG. 2 is a schematic diagram of the apparatus of FIG. 1 showing transmission, scattering and detection of a second detection beam passing through the object;

FIG. 3 is a schematic diagram of another apparatus for performing tomography, which includes a laser and a movable mirror for sweeping a detection beam across an object;

FIG. 4 is a schematic diagram of another apparatus for performing tomography, which includes a laser, a movable mirror, and an actuator for sweeping a detection beam across an object;

FIG. 5 is a schematic diagram of another apparatus for performing tomography, which includes a Fresnel lens downstream of the object being scanned;

FIG. 6 is a schematic diagram of another apparatus for performing tomography, which includes a detector, a strip of diffusive material, and a strip of Fresnel lens configured to move in-line with movement of the detection beam;

FIG. 7 is a flow chart illustrating a method of performing tomography according to one embodiment;

FIG. 8 is a flow chart illustrating a method of calibrating radiotherapy equipment according to one embodiment; and

FIGS. 9A and 9B are images of slices from a 3D reconstruction of scans taken using an apparatus made in accordance with one or more of the embodiments described herein.

### DETAILED DESCRIPTION

According to some aspects, a method of calibrating radiotherapy equipment is provided. The method includes: obtaining a reference image of a dosimeter using a tomography method comprising scanning the dosimeter by sweeping a detection beam across a plurality of segments of the dosimeter; scattering a transmitted portion of the detection beam that passes through the dosimeter to in order to generate a scattered signal, the scattered signal being generated by directing the transmitted portion across a diffuser having a diffusive surface area; and for each of the plurality of segments, detecting a portion of the scattered signal, the portion of the scattered signal being detected over a total detection area that is smaller than the diffusive surface area; after obtaining the reference image, irradiating the dosimeter with a test dosage of radiation from the radiotherapy equipment; after irradiating the dosimeter with the test dosage, obtaining a calibration image of the dosimeter using the tomography method of step (a); and comparing the reference image and the calibration image to model effects of the test dosage of radiation on the dosimeter.

According to another aspect, a method of performing tomography comprising: scanning an object with a detection beam; scattering a transmitted portion of the detection beam that passes through the object in order to generate a scattered signal, the scattered signal being generated by directing the transmitted portion across a diffuser having a diffusive surface area; and detecting a portion of the scattered signal, the portion of the scattered signal being detected over a total detection area that is smaller than the diffusive surface area.

In some embodiments, the scattering step converts the transmitted portion of the detection beam from a narrow beam to the scattered signal, the scattered signal having a lower intensity than the narrow beam.

In some embodiments, the scanning step includes sweeping the detection beam across the object to scan a plurality of segments of the object.

In some embodiments, the scanning step includes: emitting the detection beam towards a mirror; and moving the mirror to a plurality of positions, each position being selected to reflect the detection beam towards a respective segment of the object to determine transmissivity of the respective segment of the object. In some embodiments, the mirror is moved by rotating the mirror.

In some embodiments, the method further comprises correlating the detected portion of the scattered signal with each position of the detection beam in order to determine the transmissivity of each segment of the object that the detection beam passes through.

In some embodiments, the scanning step is performed using a raster scanning technique.

In some embodiments, the diffuser includes a diffusive screen defining the diffusive surface area.

In some embodiments, the method further comprises immersing the object within a liquid having a similar refractive index as the object.

In some embodiments, the object has a nominal size of at least 10 centimeters.

In some embodiments, the transmitted portion of the detection beam does not pass through an optical lens before being detected.

In some embodiments, the total detection area is configured so that a majority of the scattered signal is not detected.

In some embodiments, the method further comprises scanning the object along a plurality of projections to generate a three-dimensional image.

In some embodiments, the method further comprises rotating the object after scanning the object along an initial projection so as to begin scanning the object along a subsequent projection.

According to another aspect, an apparatus for performing tomography, the apparatus comprising: an emitter for scanning an object with a detection beam; a diffuser for scattering a transmitted portion of the detection beam that passes through the object in order to generate a scattered signal, the diffuser having a diffusive surface area; and at least one detector for detecting a portion of the scattered signal, the at least one detector having a total detection area that is smaller than the diffusive surface area.

In some embodiments, the diffuser is configured to convert the transmitted portion of the detection beam from a narrow beam to the scattered signal, the scattered signal having a lower intensity than the narrow beam.

In some embodiments, the emitter includes: a laser configured to emit the detection beam; and a moveable mirror for sweeping the detection beam across the object.

In some embodiments, the emitter includes: a laser configured to emit the detection beam; a rotatable mirror for sweeping the detection beam across the object in a fan-shaped pattern; and an actuator for translating at least one of the laser, the rotatable mirror, and the object such that the fan-shaped pattern can be used to scan the object in a plurality of linear segments.

In some embodiments, the emitter is configured to perform raster scanning of the object.

In some embodiments, the emitter emits the detection beam along a beam path, and wherein the diffuser is located along the beam path between the object and the detector.

In some embodiments, the at least one detector has a fixed location.

In some embodiments, the at least one detector is a single detector defining the total detection area.

In some embodiments, the diffuser includes a first diffusive screen defining the diffusive surface area.

In some embodiments, the diffuser includes a second diffusive screen arranged in series with the first diffusive screen.

In some embodiments, the apparatus further comprises a condensing optical lens arranged in series with the first diffusive screen.

In some embodiments, the condensing optical lens is a Fresnel lens.

In some embodiments, the condensing optical lens is located upstream of the diffusive screen.

In some embodiments, the transmitted portion of the detection beam does not pass through an optical lens before being detected.

In some embodiments, the total detection area is configured so that a majority of the scattered signal is not detected.

In some embodiments, the apparatus further comprises a container that is at least partially transparent to the detection beam, the container defining a chamber for receiving the object to be scanned along with a liquid having a similar refractive index as the object with respect to the detection beam.

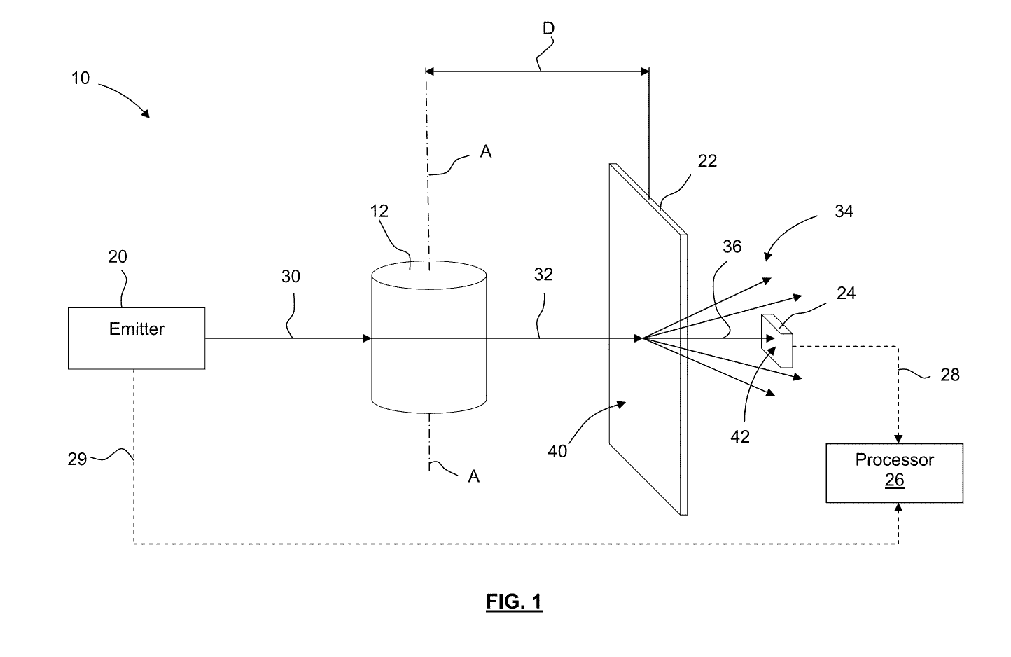

Referring to FIG. 1, illustrated therein is an apparatus 10 for performing tomography of an object 12 such as a dosimeter used in calibrating radiotherapy equipment. The apparatus 10 generally includes an emitter 20, one or more detectors 24, and at least one diffuser 22 positioned between object 12 and the detector 24.

In use, the emitter 20 scans the object 12 with a detection beam 30, which can have various scan patterns as discussed below.

When the detection beam 30 impinges the object 12, portions of the detection beam 30 may be adsorbed, reflected and/or transmitted.

A transmitted portion 32 of the detection beam 30 passes through the object 12 toward the diffuser 22. The diffuser 22 scatters the transmitted portion 32 of the detection beam 30 in order to generate a scattered signal 34. The detector 24 then detects a portion 36 of the scattered signal 34.

As shown in the illustrated embodiment, the diffuser 22 has a diffusive surface area 40, and the detector 24 has a total detection area 42 that is smaller than the diffusive surface area 40.

One benefit of the apparatus 10 is that the detector 24 can be much smaller than the size of the object being scanned 12, while still allowing the object to be scanned across multiple points or segments. This is because the diffuser 22 scatters the transmitted portion 32 of the detection beam 30 and redirects a portion 36 of the transmitted portion 32 toward the detection area 42. In other words, the diffuser 22 can convert the transmitted portion 32 of the detection beam 30 from a narrow beam having a high intensity, to a scattered signal 34 having a lower intensity. A portion 36 of this scattered signal 34 can then be detected by a small detector 24 that may be offset from the path of the detection beam 30.

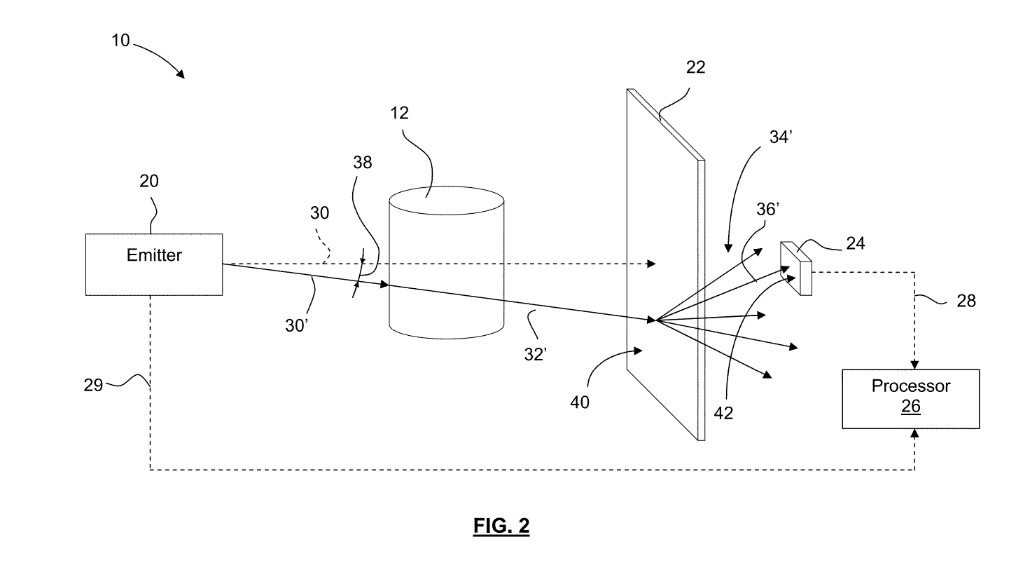

For example, with reference to FIG. 2, the emitter 20 is shown emitting a second detection beam 30′ that is at an angle 38 to the first detection beam 30 (and offset from the detector 24). The diffuser 22 scatters a transmitted portion 32′ of the second detection beam 30′ in order to generate a scattered signal 34′, and the detector 24 detects a portion 36′ of that scattered signal 34′. Specifically, as shown, the diffuser 22 scatters the transmitted portion 32′ so that a portion 36′ of the scattered signal 34′ is redirected towards the detector 24, even though the second detection beam 30′ was at an angle 38 that that would not have otherwise intersected with the detector 24.

Redirecting a portion 36 of the scattered signal 34 towards the detector 24 allows the use of a relatively small detector 24 as compared to the size of the object 12 being scanned. Reducing the size of the detector 24 can be particularly useful because it avoids the need for large detectors or large optical lenses (that can be expensive) when scanning large objects (e.g., dosimeters having diameter of 10 cm or more). In some cases, the apparatus 10 may be particularly beneficial for scanning objects having a nominal size of greater than 10-centimeters, or more particularly, greater than 30-centimeteres.

Using the diffuser 22 to redirect the portion 36 of the scattered signal 34 towards the detector 24 can also avoid the need to maintain alignment of the detector 24 and the detection beam 30. For example, it can be difficult to move the detector 24 in-line with movement of the detection beam 30 to capture or intercept the transmitted portion 32 while performing scan, particularly at high speeds. With the diffuser 22, such movement of the detector 24 is not necessary. However, in some embodiments, it may still be desirable to move the detector 24.

Referring again to FIG. 1, the emitter 20 is generally selected so that a portion of the detection beam 30 is at least partially transmitted through the object 12. For example, the emitter 20 may include a laser for scanning the object 12 with a beam of light. The use of visible light can be suitable for performing optical tomography on dosimeters that model soft tissues such as skin, muscles, nerves, fat, and the like.

While visible light has been described, the emitter 20 could also scan the object 12 with other forms of electromagnetic radiation, such as microwaves, infrared rays, terahertz rays, ultraviolet rays, X-rays, gamma rays, and the like. For example, the emitter 20 could use X-rays to scan bones and other objects that might not transmit visible light.

The emitter 20 may be configured to emit the detection beam 30 along one or more beam paths. For example, as shown in FIGS. 1 and 2, the emitter emits a first detection beam 30 along a first beam path, and a second detection beam 30′ along a second beam path that is different from the first beam path. Scanning the object 12 along multiple beam paths can allow different segments of the object to be scanned individually, and the detected measurements can be compiled to form an image.

In some embodiments, a large number of beam paths may be used to provide a detailed measurement of a large number of points of the object 12. Furthermore, the beam paths may be selected to perform cone beam reconstruction or fan beam reconstruction as will be described below.

While multiple beam paths can be used, the apparatus 10 may be configured so that only one beam or ray passes through the object 12 at a time. This can reduce cross-talk associated with multiple beams (e.g. reduce stray light), and thereby, increase accuracy of images recorded.

As shown, the diffuser 22 is located between the object 12 and the detector 24. Furthermore, the diffuser 22 is normally configured to be located along each of the beam paths (although in some cases beam paths at very large angles may not be captured). More specifically, the diffusive surface area 40 may be sized, shaped, and positioned so that the transmitted portions 32, 32′ of the detection beams 30, 30′ impinge upon the diffuser 22 so they can be scattered and redirected towards the detector 24.

For example, the diffusive surface area 40 may be sized and shaped to be at least as large as the nominal size of the object 12 being scanned. In some embodiments, the diffusive surface area 40 may be at least as large a projection of the object 12 on a plane that is located at a distance D downstream from the object 12 (e.g., corresponding to the distance between object 12 and the diffuser 22).

As shown, the diffuser 22 may include a diffusive screen that defines the diffusive surface area 40. The diffusive screen may be made from plastic such as white Mylar™ film or another material selected to diffuse the electromagnetic radiation of the detection beam 30. In some examples, the diffuser 22 could also include another type of diffusive element such as a filter made from a sheet of glass or plastic. Furthermore, in some examples, white opaque diffusers may be inclined at an angle (e.g. 45-degrees); however, this may result in reduced signal quality with respect to forward scatter of the transmitted portion 32 of the detection beam 30.

The detector 24 is generally a sensor that measures light or other electromagnetic energy based on the type of detection beam being used. Furthermore, the detector 24 may be highly sensitive in order to detect small portions 36 of the scattered signal 34 that impinge the detector 24. For example, in some embodiment the detector 24 may be a photomultiplier tube or a photodiode. More specifically, the detector 24 may be a solid-state photomultiplier or avalanche photodiode.

In some examples, the detector 24 may have a fixed location downstream of the diffuser 22. In other examples, the detector 24 may be moved with the detection beam 30 as it is swept across the object 12. For example, an actuator may be configured to move the detector 24 and the diffuser 22 concurrently or simultaneously with movement of the detection beam 30.

In some examples, the detector 24 may be a single detector defining the total detection area 42. In other examples, there may be a plurality of detectors (e.g., arranged in a detector array), with each detector having its own detection area so that the sum of the plurality of detectors defines the total detection area 42.

As described above, the detection area 42 is generally smaller than the diffusive surface area 40, and in some cases, significantly smaller. As such, a majority of the scattered signal 34 is normally not detected by the detector 24.

While this might attenuate the signal measured by the detector 24, some high-sensitivity detectors such as photomultipliers and photodiodes are still capable of obtaining meaningful data from the portion 36 of the scattered signal 34 that impinges the detector 24. Thus, it is possible to calculate and determine the opacity or transmissivity of each particular segment of the object 12 that the detection beam 30 passes through.

In some examples, it may be desirable to use a detector 24 having some minimum surface area. Using a detector 24 having a detection area 42 greater than some minimum size may allow spatial averaging of the amount of transmitted radiation detected. This may reduce the effects of speckle associated with a particular form of electromagnetic radiation by averaging out noise due to speckle. In some examples, the detection area 42 may be greater than about 20-cm², or more particularly, greater than about 40-cm². In other examples, the detection area 42 may be larger or smaller.

In some examples, the diffuser 22 may be configured to counteract the effects of noise or speckle in other ways. For example, the diffuser 22 may be spaced apart further from the object 12 to obtain a wider beam for spatial averaging on the diffuser 22. Emitting a wider detection beam 30 from the emitter 20 can also avoid noise. Furthermore, it might be possible to reduce noise or speckle by using diffusers with finer structures, or providing a set of diffusers arranged in series.

Referring still to FIG. 1, the apparatus 10 may include a processor 26 which could be a personal computer, a dedicated microprocessor, an electronic circuit, or another type of computing device. The processor 26 may be configured to generate an image or a model of the object 12 based on measurements from the detector 24. For example, the processor 26 may be configured to receive measurement data 28 from the detector 24, and use the measurement data 28 to calculate the transmissivity of the particular segment of the object 12 that the detection beam 30 passed through.

Furthermore, the processor 26 can receive position information 29 from the emitter 20 corresponding to the position of the detection beam 30. The processor 26 can then correlate the position information 29 with the calculated transmissivity from the measurement data 28 in order to generate an image or model of the object 12 by scanning multiple segments. The processor 26 could also be configured to operate the emitter 20 to control the position of the detection beam 30 for scanning segments of the object 12.

In some embodiments, the apparatus 10 may be configured to perform three-dimensional imaging. For example, a plurality of projections can be taken by scanning the object 12 along a first projection, rotating the object 12 relative to the apparatus 10 (e.g. about an axis of rotation A), or rotating the emitter 20 (or both), and then rescanning the object 12 along a second projection.

After rotating and rescanning the object 12 a number of times, the recorded measurements taken at each projection can be reconstructed to generate a three-dimensional model of the object 12.

In some examples, the processor 26 may be configured to perform cone beam reconstruction, fan beam reconstruction, or another type computed tomography.

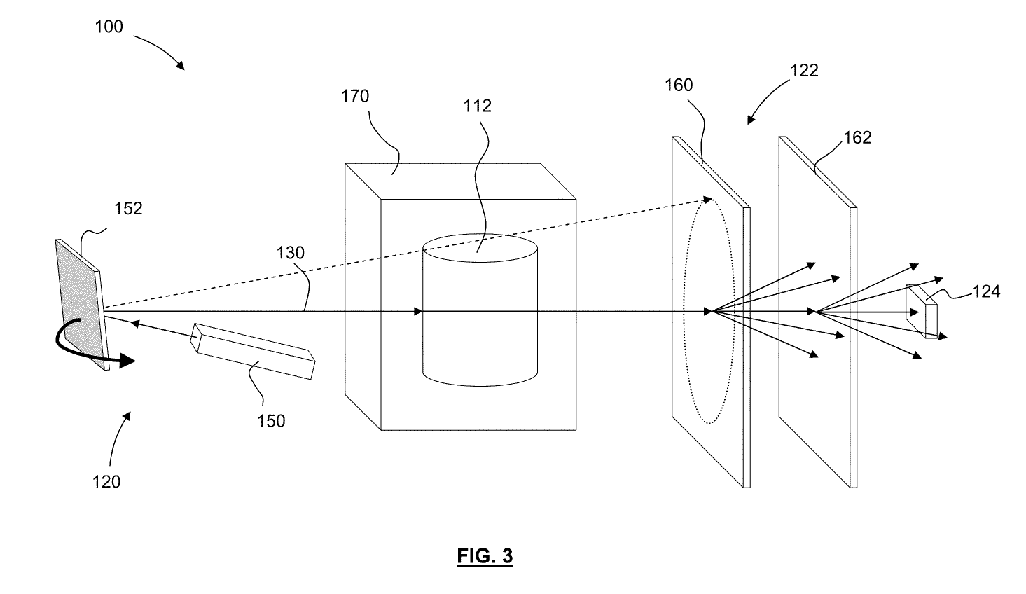

Referring now to FIG. 3, illustrated therein is another example of an apparatus 100 for performing tomography of an object 112. The apparatus 100 is similar in some respects to the apparatus 10 and where appropriate similar elements are given similar reference numerals incremented by one hundred. For example, the apparatus 100 includes an emitter 120, a diffuser 122, and a detector 124.

In this embodiment, the emitter 120 includes a laser 150 configured to emit a detection beam 130, such as a yellow He-NE laser or another suitable laser.

The spatial resolution of each scan is generally based on the diameter of the detection beam 130 when it impinges the object 112.

In some examples, the full-width, half-maximum of the beam may be less than about 2-millimeters, or more particularly, less than about 1-millimeter, or more particularly still, about 0.6-millimeters or smaller. Generally speaking, better resolution can be obtained using narrower beams. However, wider beams can provide faster scan times. Accordingly, larger or smaller beams may be used for various scans depending on whether greater resolution or faster scan times are more desirable.

In this embodiment, the emitter 120 also includes a movable mirror 152 (e.g., a rotatable mirror) for moving the detection beam 130 across the object 112 in a particular pattern such as a cone-shaped pattern. For example, the mirror 152 may be part of a two-dimensional mirror system such as the “GVSM002/M-2D Galvo System” produced by Thorlabs™.

This type of mirror system can allow raster scanning, for example, when performing cone beam reconstruction. Specifically, the object 112 can be scanned point-by-point in lines. For example, the mirror 152 can be rotated along a yaw-axis to sweep the detection beam 130 horizontally across each line while measuring transmissivity of each point through the object. After completing one horizontal line, the mirror 152 can be rotated about a pitch-axis to move the detection beam 130 vertically to the next horizontal line and begin sweeping the detection beam 130 across the next set of points. Scanning point-by-point can provide high resolution images. Moreover, a narrower collimated detection beam can provide a large depth of focus (e.g. with a uniform resolution throughout the object 12).

In some embodiments, non-uniform or non-linear scan patterns may be used to scan an area of interest on the object. This can increase the resolution of that area, which can be particularly beneficial for portions of the object that have low transmissivity.

While mirrors have been described, it may be possible to sweep the detection across the object using other techniques. For example, it may be possible to perform electro-optic deflection of the detection beam using a crystal.

Referring still to FIG. 3, the diffuser 122 may include two diffusive screens 160, 162 arranged in series. The diffusive screens 160, 162 may be made from the same material or different materials. The use of two or more diffusive screens (or other diffusive elements) arranged in series can enhance scattering of the transmitted portion of the detection beam and may enhance spatial averaging. Using two or more diffusers might also reduce speckle or other forms of noise.

As shown, the apparatus 100 may also include a container 170. The container 170 defines a chamber for receiving the object 112 to be scanned. The container 170 is at least partially transparent so that the detection beam 130 can pass through the container to the object 112, in some embodiments without substantial interference with the detection beam 130.

In some embodiments, the container 170 may be filled with a liquid. The liquid may help stabilize and hold the object 112 in place while performing a scan. This can be particularly helpful when scanning a deformable object that might not be able support its own weight (such as a deformable dosimeter). Furthermore, the liquid may be selected to have a similar refractive index as the object 112, and which may be equivalent to some type of tissue. For example, the liquid may be water when scanning dosimeters modeling human tissues. Matching the refractive index of the object 112 and the liquid can reduce artifacts and other distortions while performing a scan. Furthermore, the liquid can improve scanning around the edges of the object 112, for example, by allowing more data to be collected which can provide more accurate imaging.

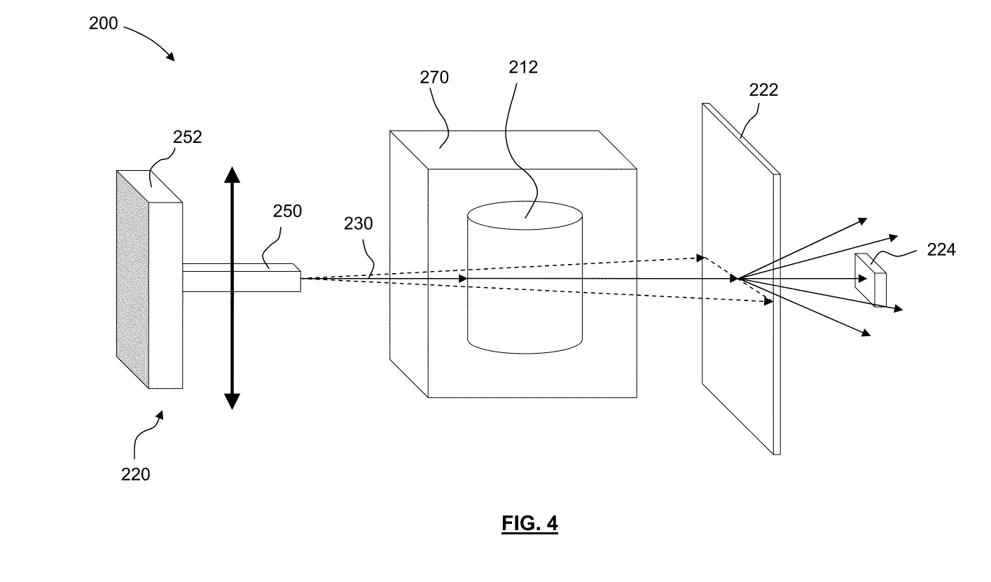

Referring now to FIG. 4, illustrated therein is another example of an apparatus 200 for performing tomography of an object 212. The apparatus 200 is similar in some respects to the apparatus 100 and where appropriate similar elements are given similar reference numerals incremented by one hundred. For example, the apparatus 200 includes an emitter 220, a diffuser 222, a detector 224, and a container 270.

In the illustrated embodiment, the emitter 220 includes a laser 250 configured to emit a detection beam 230 and scan the object 212 across a fan-shaped pattern (i.e. to scan the object in linear segments). For example, the laser 250 may emit a narrow detection beam and a rotatable mirror (not shown) may sweep the detection beam 230 across the object 212 in a fan-shaped pattern. This can allow fan beam reconstruction of the object 212, which can provide isotropic spatial resolution and faster scan times in comparison to cone beam reconstruction.

The emitter 220 also includes an actuator 252 for translating the laser 250 (and/or the rotatable mirror). This can allow the detection beam 230 to move to the next linear segment when performing fan beam reconstruction. The actuator 252 may be a linear motor, a rotating screw driven by a rotary motor, a hydraulic or pneumatic cylinder, and the like.

In other examples, the detection beam 230 could be moved to the next line using other techniques, for example, by rotating or otherwise moving a mirror.

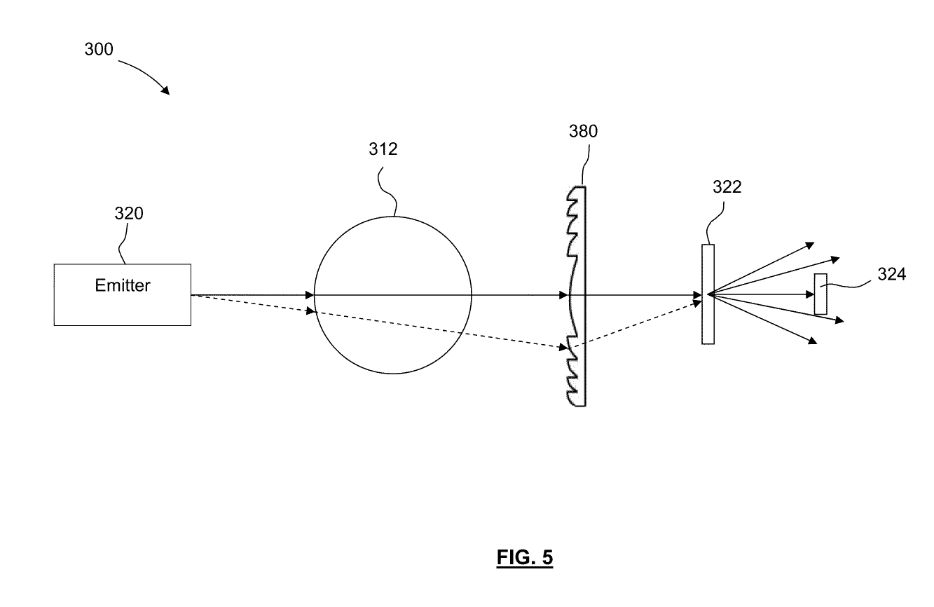

Referring now to FIG. 5, illustrated therein is another example of an apparatus 300 for performing tomography of an object 312. The apparatus 300 is similar in some respects to the apparatus 10 and where appropriate similar elements are given similar reference numerals incremented by three hundred. For example, the apparatus 300 includes an emitter 320, a diffuser 322, and a detector 324.

The apparatus 300 includes a condensing optical lens 380 such as a Fresnel lens. The condensing optical lens 380 is arranged in series with the diffuser 322, which in this case, includes a single diffusive screen.

As shown, the condensing optical lens 380 is located upstream of the diffusive screen. The condensing optical lens 380 may focus transmitted portions of the detection beam onto the diffuser 322. This can provide more efficient collection of light, and can allow the use of a diffuser 322 with a smaller diffusive surface area. For example, the diffusive surface area may be sized slightly larger than the anticipated wander of the transmitted portion of the detection beam in a plane aligned with the diffuser 322.

In general, the use of the condensing optical lens 380 can provide more uniform intensity of the scattered signal. However, the condensing optical lens 380 may increase the cost of the apparatus, and might also introduce artifacts such as reflections or other interference effects.

In some embodiments, it may be desirable to omit the condensing optical lens 380. More specifically, it may desirable to configure the apparatus so that the transmitted portion of the detection beam does not pass through any condensing or other optical lenses before being detected. Omitting the optical lenses may provide higher resolution images.

In some examples, the diffuser 322 may be omitted and the condensing optical lens 380 may focus the transmitted portions of the detection beam directly onto the detector 324.

In yet other examples, an opaque screen with an aperture may be placed between the condensing optical lens 380 and the detector 324.

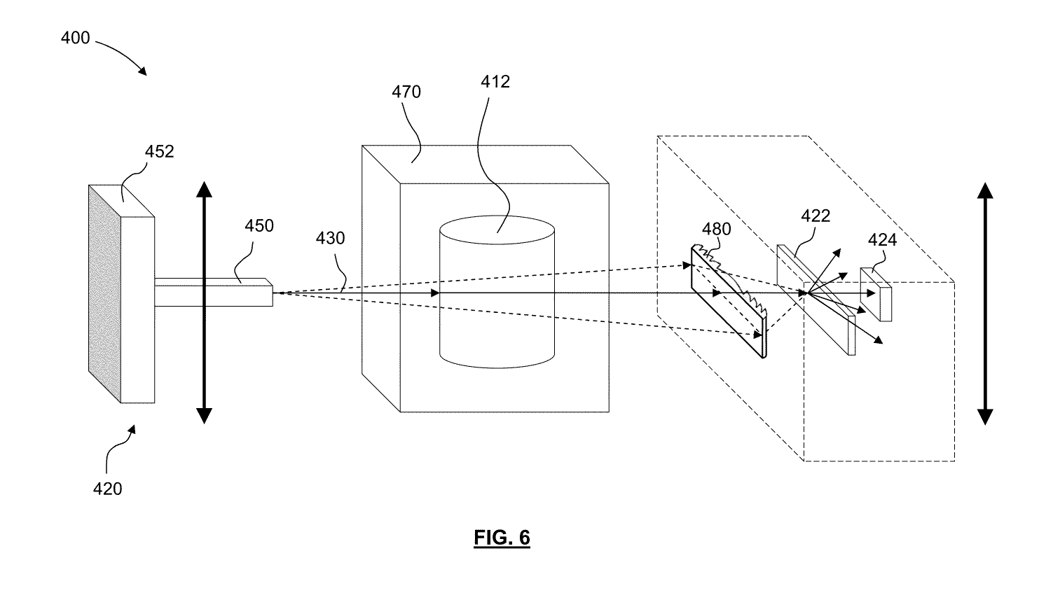

Referring now to FIG. 6, illustrated therein is another example of an apparatus 400 for performing tomography of an object 412. The apparatus 400 is similar in some respects to the apparatus 200 and where appropriate similar elements are given similar reference numerals incremented by two hundred. For example, the apparatus 400 includes an emitter 420 (including a laser 450 and an actuator 452 for moving a detection beam 430 across the object 412 in a fan-shaped pattern), a diffuser 422, a detector 424, and a container 470.

In this embodiment, the diffuser 422 and the detector 424 are configured to translate or otherwise move in-line with the detection beam 430. For example, the diffuser 422 and the detector 424 may be mounted to a module that moves with the laser 450 when translated by the actuator 452. The module may be moved by the same actuator 452 that moves the laser 450, or second independent actuator.

Moving the diffuser 422 and the detector 424 with the detection beam 430 can allow the diffuser 422 to be made with a shorter height than the diffuser 222.

The apparatus 400 may also include a strip of condensing lens 480 such as a strip of Fresnel lens. The lens 480 may focus transmitted portions of the detection beam onto the diffuser 322 and can provide more uniform intensity of the scattered signal. The condensing lens 480 may also be mounted to the same module as the diffuser 422 and the detector 424 so as to move with the detection beam 430.

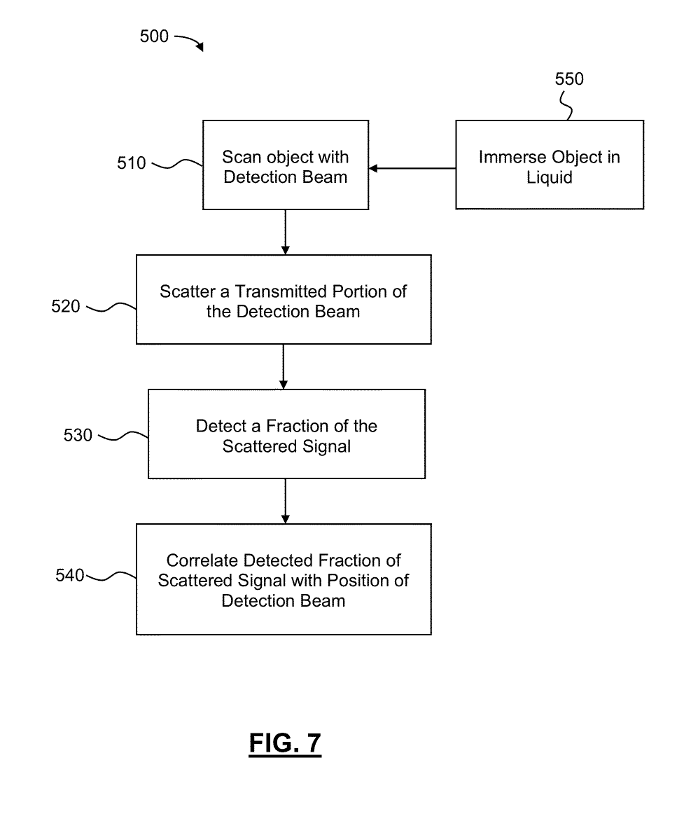

Referring now to FIG. 7, illustrated therein is a method 500 of performing tomography according to one embodiment. The method generally includes steps 510, 520, and 530, although other steps may also be performed (e.g., steps 540 and 550).

Step 510 includes scanning an object with a detection beam. In some examples, the object being scanned may have a nominal size of at least 10 centimeters, or more particularly, at least 30-centimeters. In some examples, the object may be a dosimeter.

The scanning step 510 may include sweeping the detection beam across the object to scan a plurality of segments of the object. For example, the detection beam may be swept across the object by emitting the detection beam towards a mirror, and moving the mirror to a plurality of positions. Each position of the mirror may be selected to reflect the detection beam towards a particular segment of the object to determine the transmissivity of that segment. For example, the detection beam may be moved across the object point-by-point and line-by-line. This type of scan may be referred to as a raster scan, and may be performed (for example) using the laser 150 and movable mirror 152 of the emitter 120. The raster scan can be used to perform cone beam reconstruction.

In other examples, the detection beam may be swept across the object using other techniques. For example, the scanning step 510 may include emitting a detection beam towards the object, and sweeping the detection beam across the object in a fan-shaped pattern (e.g. using a rotatable mirror). The detection beam can then be translated to the next line (e.g. using an actuator). This type of scan may allow fan beam reconstruction and can be performed (for example) using the laser 250 and the actuator 252 of the emitter 220.

Step 520 includes scattering a transmitted portion of the detection beam that passes through the object in order to generate a scattered signal. For example, the transmitted portion of the detection beam may be directed towards a diffuser such as one of the diffusers 22, 122, 222, 322, 422 described above. The diffuser generally has a diffusive surface area for scattering the transmitted portion of the detection beam.

Step 530 includes detecting a portion of the scattered signal. More specifically, the portion of the scattered signal is detected over a total detection area that is smaller than the diffusive surface area. For example, the portion of the scattered signal may be detected using one of the detectors 24, 124, 224, 324, 424 described above.

As described previously, the detection area is generally configured so that a majority of the scattered signal is not detected.

In some embodiments, the detecting step 530 may be carried out so that the transmitted portion of the detection beam does not pass through an optical lens before being detected.

In some embodiments, when the scanning step 510 includes sweeping the detection beam across the object to scan a plurality of segments of the object, the method 500 may also include step 540 of correlating the detected portion of the scattered signal with each position of the detection beam in order to determine the transmissivity of each segment of the object through which the detection beam had passed. In some embodiments, the correlating step 540 may be performed using the processor 26. Furthermore, the processor 26 may also be configured to generate an image or model based on a plurality of scans.

Step 510 may be performed so that only one beam or ray passes through the object at a time. This may reduce cross-talk associated with multiple beams and can increase accuracy of images reconstructed using the method 500.

In other examples, a plurality of beams may be emitted towards the object concurrently. Each beam may be aimed at a different segment of the object and may be frequency modulated or otherwise encoded to differentiate the beams from one another. Portions of each beam may then be detected and reconstructed to form an image based on the position and encoding information for each beam. Using more than one detection beam at a time can increase scanning speeds.

The method 500 may also include step 550 of immersing the object within a liquid having a refractive index that is similar to the refractive index of the object. As shown, step 550 generally occurs before step 510. Immersing the object within the liquid may help stabilize and hold the object in place while performing a scan.

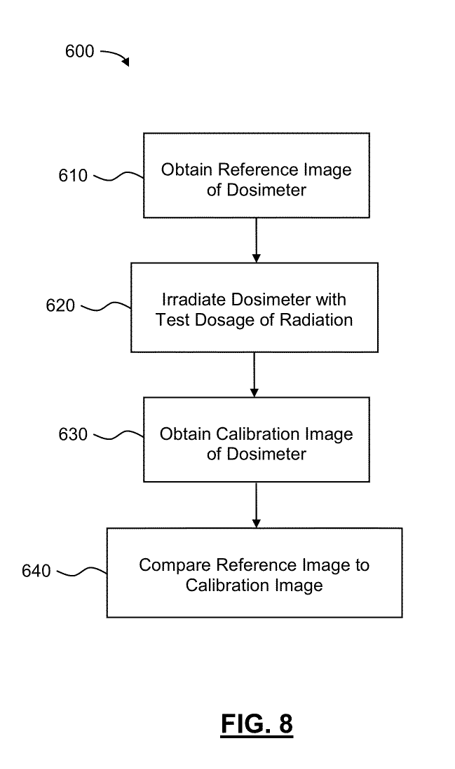

Referring now to FIG. 8, illustrated therein is a method 600 of calibrating radiotherapy equipment according to one embodiment. The method includes steps 610, 620, 630, and 640, although in some cases other steps may also be performed.

Step 610 includes obtaining a reference image of a dosimeter using a tomography method. The tomography method may be similar to the method 600 described above, and which may use one or more of the apparatuses as generally described herein.

For example, the tomography method may include scanning the dosimeter by sweeping a detection beam across a plurality of segments of the dosimeter, scattering a transmitted portion of the detection beam that passes through the dosimeter in order to generate a scattered signal, and, for each of the plurality of segments, detecting a portion of the scattered signal. The scattered signal is generally generated by directing the transmitted portion across a diffuser having a diffusive surface area. Furthermore, the portion of the scattered signal is detected over a total detection area that is smaller than the diffusive surface area.

The dosimeter is generally made of a material that experiences a change in optical transmittance (e.g., turns opaque) when irradiated with a particular type of ionizing radiation such as X-rays or gamma-rays. However, the detection beam used in the tomography method is generally a different type of electromagnetic radiation that would not affect the optical transmittance of the dosimeter. For example, the detection beam may include visible light, microwaves, infrared rays, ultraviolet rays, terahertz rays, and the like.

In some examples, step 610 may include rotating the dosimeter to obtain a plurality of reference images along a number of projections. For example, step 610 may include obtaining a first reference image by scanning the dosimeter along a first projection, rotating the dosimeter (or the source of the detection beam), and then rescanning the dosimeter along a second projection. After rotating and rescanning the dosimeter a number of times, the reference images taken at each projection can be reconstructed to generate a three-dimensional model of the dosimeter.

Step 620 includes irradiating the dosimeter with a test dosage of radiation from the radiotherapy equipment. The test dosage may be a form of ionizing radiation such as X-rays or gamma rays. The test dosage may be applied based upon a treatment plan for a particular patient.

In some embodiments, this step 620 may include removing the dosimeter from the tomography apparatus, and then placing the dosimeter into the beam path of the radiotherapy equipment.

Step 630 occurs after irradiating the dosimeter with the test dosage of radiation and includes obtaining a calibration image of the dosimeter using a tomography method such as the same tomography method used at step 610. This step 630 may include returning the dosimeter to the tomography apparatus.

In some examples, step 630 may include rotating the dosimeter to obtain a plurality of calibration images along a number of projections, for example, by scanning, rotating and rescanning, the dosimeter as in step 610.

Step 640 includes comparing the reference image and the calibration image to model effects of the test dosage of radiation on the dosimeter. Generally, the test dosage of radiation applied at step 620 ionizes parts of the dosimeter, which can change the opacity/transmittance of the dosimeter at those points. Accordingly, the calibration image can be used to model the effects of the test dosage of radiation by locating the points on the model having increased opacity.

With this information, a determination can be made (e.g., by a medical practitioner, automatically using software, etc.) as to whether the test dosage corresponds to the treatment plan prescribed for a patient. If not, adjustments or recalibrations can be made to the radiotherapy equipment in order to apply the appropriate dosage.

In some examples, the comparison of the reference image to the calibration image can be used to perform computed tomography reconstruction such as cone beam reconstruction or fan beam reconstruction.

While the embodiment described above relates to dosimeters, in other embodiments an object may be imprinted with a 3D image using ionizing radiation or another form of energy, and the object may be scanned using the method 600 described above to identify or read the 3D image imprinted within the object.

Furthermore, while some embodiments refer to irradiating the object with ionizing radiation, the object could be configured to experience a change in transmittance when subjected to another form of energy such as ultrasound, light, heat, magnetic fields, and the like.

### Experiments

Experiments were performed using one or more of apparatus that were generally similar to the apparatus described herein. Specifically, an apparatus generally similar to the apparatus 100 shown in FIG. 3 was used to scan samples using the tomography method described. The apparatus included a yellow He—Ne laser (attenuated to ˜10 microwatts) fitted with a spectral filter (10 nm bandpass, central wavelength of 594 nanometers) and a 1-millimeter diameter spatial filter, a 2D scanning galvo-mirror system made by Thorlabs™ under model number GVSM002/M, a container filled with water and having a field of view of 18-centimeters, a first diffusive screen made from white Mylar film, a second diffusive screen made of white plastic sheet, and a photomultiplier tube (PMT) as a detector. The current from the PMT was amplified using a proprietary electronic circuit and digitized using a 12-bit data acquisition card within a personal computer. A MATLAB™ program was developed for cone-beam reconstruction using a graphics processing unit made by Nividia™ and used CUDA routines.

The samples being scanned were polyethylene terephthalate containers filled with aqueous carbon black solutions using Triton X100 and hydrogen peroxide as an anti-microbial agent. The containers had a diameter of 15-centimeters and were supplied by Modus Medical Devices Inc. Absorption coefficients were measured independently with a visible light absorption spectrometer made by Hitachi-Perkin Elmer under Model 204.

The apparatus was used to complete 512 projections over 360-degrees with a field of view of 12×18-centimeters. The scans took 30-minutes to complete. Full 3D reconstruction, including reading of input data, took less than 20 seconds for a 512×512×200 array of data.

During the experiments, a large cone of stray light (corresponding to 2% of the laser beam intensity) illuminated the liquid filled container. A stray light measurement was performed by placing a beam block at the entrance of the liquid filled container and sampling the signal in the shadow. This value was subtracted for each pixel in the projection image to filter out the stray light component. The source of this stray light is believed to be scatter from the galvo-mirror system. Mirror replacement may reduce this stray light component.

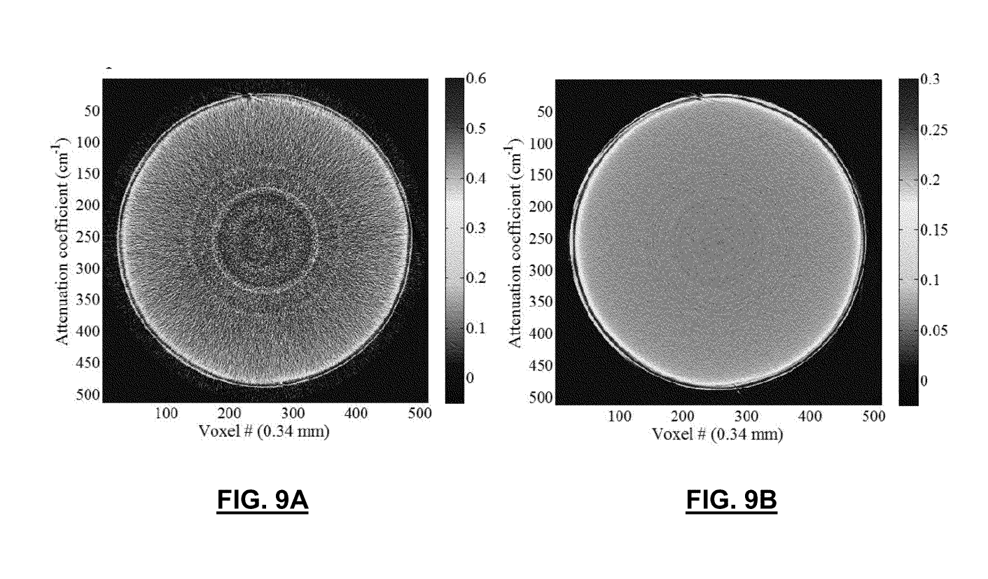

FIGS. 9A and 9B show central slices from two different samples. The sample in FIG. 9A included a dark object with a minimum transmission of 0.1%, and the sample in FIG. 9B included an intermediate opacity object with a minimum transmission of 4%. With reference to FIG. 9A, there are a number of artifacts in the shape of rings toward the center of the samples. It is believed that these rings are caused by reflections within the liquid filled container. It may be possible to reduce or eliminate these rings by adding antireflective windows and flat black surfaces to the container.

In general, averaged reconstruction coefficients were within 2% along the height of the sample and within the central 85% of diameter. It may be possible to improve this by providing better refractive index matching with the container. Agreement with spectrometer measurements was better than 0.5% for the lighter sample having a transmission of 4%, and within 4% for the dark sample having a transmission of 0.1%. Specifically, mean attenuation coefficients for the solutions measured with spectrometer and the apparatus, were 0.220 cm⁻¹ and 0.220 cm⁻¹ for the first sample, and 0.453 cm⁻¹ and 0.473 cm⁻¹ respectively. An error in the stray light estimate for the dark solution may be the cause of the attenuation being greater than the spectrometer measurement.

Some embodiments of the apparatus, systems, and methods described herein may be implemented in hardware or software, or a combination of both. For example, some embodiments may be implemented in computer systems and computer programs, which may be stored on a physical computer readable medium, executable on programmable computers each comprising at least one processor, a data storage system (including volatile and non-volatile memory and/or storage elements), at least one input device (e.g. a keyboard or mouse), and at least one output device (e.g. a display screen, a network, or a remote server). For example and without limitation, the programmable computers may include personal computers, laptops, netbook computers, personal data assistants (PDA), cell phones, smart phones, gaming devices, and other mobile devices.

While the above description provides examples of one or more apparatus, methods, or systems, it will be appreciated that other apparatus, methods, or systems may be within the scope of the present description as interpreted by one of skill in the art.

1. A method of calibrating radiotherapy equipment, the method comprising:
(a) obtaining a reference image of a dosimeter using a tomography method comprising:
(i) scanning the dosimeter by sweeping a detection beam across a plurality of segments of the dosimeter;
(ii) scattering a transmitted portion of the detection beam that passes through the dosimeter to in order to generate a scattered signal, the scattered signal being generated by directing the transmitted portion across a diffuser having a diffusive surface area; and
(iii) for each of the plurality of segments, detecting a portion of the scattered signal, the portion of the scattered signal being detected over a total detection area that is smaller than the diffusive surface area;
(b) after obtaining the reference image, irradiating the dosimeter with a test dosage of radiation from the radiotherapy equipment;
(c) after irradiating the dosimeter with the test dosage, obtaining a calibration image of the dosimeter using the tomography method of step (a); and
(d) comparing the reference image and the calibration image to model effects of the test dosage of radiation on the dosimeter.

2. A method of performing tomography, the method comprising:
(a) scanning an object with a detection beam;
(b) scattering a transmitted portion of the detection beam that passes through the object in order to generate a scattered signal, the scattered signal being generated by directing the transmitted portion across a diffuser having a diffusive surface area; and
(c) detecting a portion of the scattered signal, the portion of the scattered signal being detected over a total detection area that is smaller than the diffusive surface area.

3. The method of claim 2, wherein the scattering step converts the transmitted portion of the detection beam from a narrow beam to the scattered signal, the scattered signal having a lower intensity than the narrow beam.

4. The method of claim 2, wherein the scanning step includes sweeping the detection beam across the object to scan a plurality of segments of the object.

5. The method of claim 4, wherein the scanning step includes:
(a) emitting the detection beam towards a mirror; and
(b) moving the mirror to a plurality of positions, each position being selected to reflect the detection beam towards a respective segment of the object to determine transmissivity of the respective segment of the object.

6. The method of claim 5, wherein the mirror is moved by rotating the mirror.

7. The method of claim 4, further comprising correlating the detected portion of the scattered signal with each position of the detection beam in order to determine the transmissivity of each segment of the object that the detection beam passes through.

8. The method of claim 2, wherein the scanning step is performed using a raster scanning technique.

9. The method of claim 2, wherein the diffuser includes a diffusive screen defining the diffusive surface area.

10. The method of claim 2, further comprising immersing the object within a liquid having a similar refractive index as the object.

11. The method of claim 2, wherein the object has a nominal size of at least 10 centimeters.

12. The method of claim 2, wherein the transmitted portion of the detection beam does not pass through an optical lens before being detected.

13. The method of claim 2, wherein the total detection area is configured so that a majority of the scattered signal is not detected.

14. The method of claim 2, further comprising scanning the object along a plurality of projections to generate a three-dimensional image.

15. The method of claim 14, further comprising rotating the object after scanning the object along an initial projection so as to begin scanning the object along a subsequent projection.

16. An apparatus for performing tomography, the apparatus comprising:
(a) an emitter for scanning an object with a detection beam;
(b) a diffuser for scattering a transmitted portion of the detection beam that passes through the object in order to generate a scattered signal, the diffuser having a diffusive surface area; and
(c) at least one detector for detecting a portion of the scattered signal, the at least one detector having a total detection area that is smaller than the diffusive surface area.

17. The apparatus of claim 16, wherein the diffuser is configured to convert the transmitted portion of the detection beam from a narrow beam to the scattered signal, the scattered signal having a lower intensity than the narrow beam.

18. The apparatus of claim 16, wherein the emitter includes:
(a) a laser configured to emit the detection beam; and
(b) a moveable mirror for sweeping the detection beam across the object.

19. The apparatus of claim 16, wherein the emitter includes:
(a) a laser configured to emit the detection beam;
(b) a rotatable mirror for sweeping the detection beam across the object in a fan-shaped pattern; and
(c) an actuator for translating at least one of the laser, the rotatable mirror, and the object such that the fan-shaped pattern can be used to scan the object in a plurality of linear segments.

20. The apparatus of claim 16, wherein the emitter is configured to perform raster scanning of the object.
